(19) United States
(12) Patent Application Publication
(10) Pub. No.: US 2009/0216090 A1
(43) Pub. Date: Aug. 27, 2009

(54) Household health monitoring system

(75) Inventor: Kun-Sung Chen, Miaoli City (TW)

Correspondence Address: COOPER & DUNHAM, LLP, 30 Rockefeller Plaza, 20th Floor, NEW YORK, NY, 10112, US

(73) Assignee: Sinbon Electronics Company Ltd.

(21) Appl. No.: 12/072,334

(22) Filed: Feb. 26, 2008

(51) Int. Cl. A61B 5/00

(52) U.S. Cl. 600/300

(57) ABSTRACT

A household health monitoring system has at least one medical instrument, a data gathering device and at least one electric appliance. The at least one medical instrument measures a user's measurement result information. The data gathering device stores at least one email address, is electronically connected to the at least one medical instrument to gather the measurement result information from the at least one medical instrument and sends an email having the gathered measurement result information to the at least one email address. The at least one electric appliance is used to receive the email sent to the at least one email address and showing the gathered measurement result information. Therefore, the household health monitoring system provides measurement result information of a user directly to the person designated by the user without setting a server as conventional health monitoring system.

### BACKGROUND OF THE INVENTION

1. Field of the Invention

The present invention relates to a household health monitoring system, and more particularly to a household health monitoring system that provides user's measurement result information directly to a specific person without setting a server.

2. Description of Related Art

Health monitoring system is commonly used in families having chronic members or elder members. A conventional health monitoring system comprises at least one medical instrument, a data gathering device and a server.

The medical instrument is set in a patient's home, so the family members can use it to measure health information. The data gathering device is set in the patient's home and is electronically to the medical instrument. When the data gathering device obtains the measuring results from the medical instrument, the data gathering device transforms the measuring results into measurement result information. The server may be set in a medical facility, such as a hospital, a health center or the like, and is electronically connected to the data gathering device via a communication network, such as Internet, radio transmission or the like, to receive the measurement result information. Therefore, staffs in the medical facility will examine the measurement result information to know a health condition of the patient. Accordingly, the most important benefit of the conventional health monitoring system is that the patient can be examined without meeting the medical staff face to face by using the data gathering device of the health monitoring system at home.

In addition, it is important to carefully monitor elder people's health conditions so the families having elder members can also use the conventional health monitoring system to allow the medical staff to examine the elder members' health conditions. However, elder people are not all required to be often examined health conditions by the medical staffs because not all of the elder people are sick. Compared with examining health conditions by the medical staffs, the families of the elder people intend using a new health monitoring system different from the conventional health monitoring system to directly know the elder people's measurement result information.

To overcome the shortcomings, the present invention provides a household health monitoring system to mitigate or obviate the aforementioned problems.

### SUMMARY OF THE INVENTION

The main objective of the invention is to provide a household health monitoring system.

The household health monitoring system in accordance with the present invention comprises at least one medical instrument, a data gathering device and at least one electric appliance. Each medical instrument measures a user's measurement result information. The data gathering device stores at least one email address, is electronically connected to the at least one medical instrument to gather the measurement result information from the at least one medical instrument and add the measurement result information to a new-created email created according to email addresses preset in the data gathering device. The data gathering device automatically sends the email to the preset email address so a specific person, such as a family member of the user, can receive the email from the preset email address by using an electric appliance. Therefore, as long as the user sets the email address of the specific person in the data gather device, the specific person can easily, timely and remotely receive the measurement result information of the user by using the electric appliance.

Other objectives, advantages and novel features of the invention will become more apparent from the following detailed description when taken in conjunction with the accompanying drawings.

### BRIEF DESCRIPTION OF THE DRAWINGS

FIG. 1 is a functional block diagram of a household health monitoring system in accordance with the present invention;

FIG. 2 is a functional block diagram of a data gathering device in the household health monitoring system;

FIG. 3 is a flow chart of a measurement result information notification process stored in a control unit in the data gathering device; and

FIG. 4 is a flow chart of a medicine taken reminder process stored in the control unit in the data gathering device.

### DETAILED DESCRIPTION OF PREFERRED EMBODIMENT

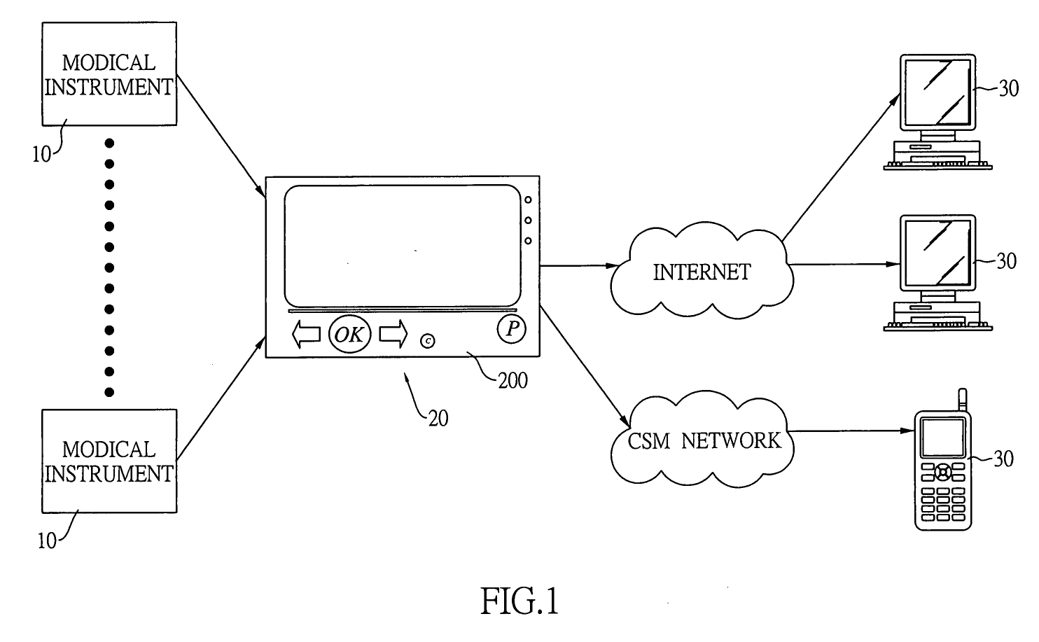

With reference to FIG. 1, a household health monitoring system in accordance with the present invention comprises at least one medical instrument (10), a data gathering device (20) and at least one electric appliance (30).

Each medical instrument (10) may be a sphygmomanometer, a clinical thermometer, a pulse oximetry device, a pulsimeter or any other physical examination or data acquisition instrument to measure a user's blood pressure, body temperature, pulse oximetry, heart rate, respiratory rate or any other measurement result information. Furthermore, the present invention may have multiple medical instruments (10).

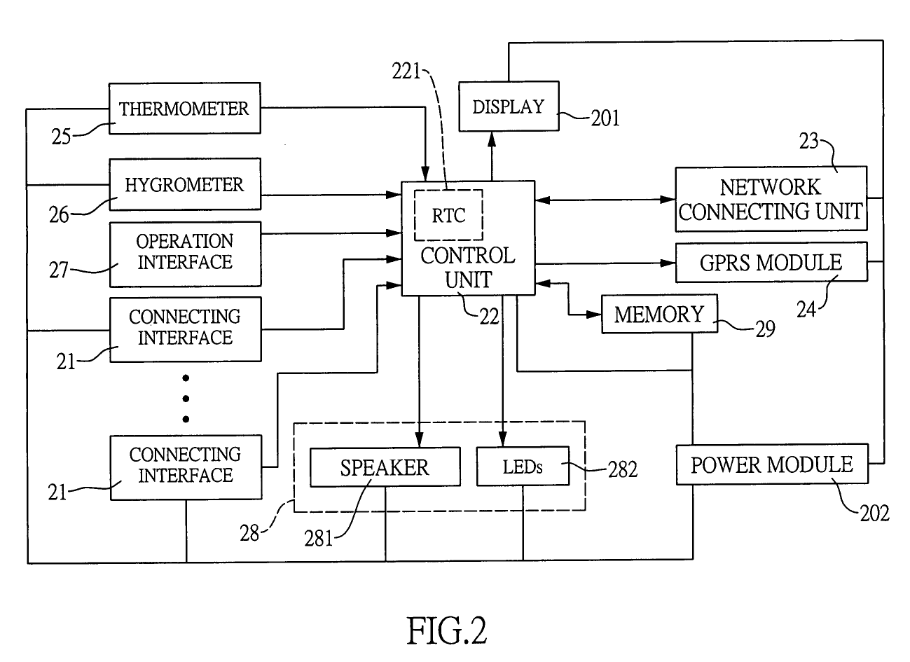

The data gathering device (20) is electronically connected to the at least one medical instrument (10) to gather the measurement result information from the medical instrument (10). With reference to FIG. 2, the data gathering device (20) further comprises a casing (200), at least one connecting interface (21), a control unit (22), a network connecting unit (23), an optional general packet radio service (GPRS) module (24), an optional thermometer (25), an optional hygrometer (26), an input device (27), a display (201), an optional indicating unit (28), a memory (29) and a power module (202).

The at least one connecting interface (21) is mounted in and exposed through the casing (200) to connect correspondingly to the at least one medical instrument (10) and may be an RS-232 interface, an universal serial bus (USB) interface, a bluetooth protocol interface, an IEEE 1394 interface or any other data transferring interface.

The control unit (22) is mounted in the casing (200) and is electronically connected to the at least one medical instrument (10) through the corresponding connecting interface (21) to gather the measurement result information from the at least one medical instrument (10). Furthermore, the control unit (22) may have analog to digital converting capability to convert analog data into digital data and a real-time clock (RTC) (221) to count the current time. When the control unit (22) gathers the measurement result information from the at least one medical instrument (10), the control unit (22) will record a measuring time corresponding to the gathered measurement result information from the RTC (211).

The network connecting unit (23) is mounted in the casing (200), is electronically connected to the control unit (22), is used to connect to the Internet and may comprise a physical wire connector (231), a wireless connector (232) or the combination therein. The physical wire connector (231) further exposes through the casing (200) to connect to the Internet with a wire and may be a network interface card. The wireless connector (232) connector connects to the Internet under wireless fidelity (Wi-Fi) protocol.

The GPRS module (24) is mounted in the casing (200), is electronically connected to the control unit (22) and is used to connect to a global system for mobile communications (GSM) network.

The thermometer (25) is mounted in the casing (200) and is electronically connected to the control unit (22) to sense and send the indoor temperature in analog data form to the control unit (22).

The hygrometer (26) is mounted in the casing (200) and is electronically connected to the control unit (22) to sense and send the indoor humidity in analog data form to the control unit (22).

The input device (27) is mounted on the casing (200), is electronically connected to the control unit (22) and may be multiple buttons to allow the user to control and operate the data gathering device (20).

The display (201) is mounted on the casing (200) and is electronically connected to the control unit (22) to display the gathered measurement result information.

The indicating unit (28) is mounted on the casing (200), is electronically connected to and is controlled by the control unit (22) and may comprise multiple light emitting diodes (LEDs) (281), a speaker (282) or the combination therein. The LEDs (281) and the speaker (282) are electronically connected to and are controlled by the control unit (22).

The memory (29) is mounted in the casing (200), is electronically connected to the control unit (22) to store the gathered measurement result information and the corresponding measuring time and stores an electric mail (email) program, at least one email address and a measurement result information notification process. The at least one email address is input by the user from operating the input device (27). Furthermore, the memory (29) may further store at least one cellphone number, at least one medicine taken reminding time, a medicine taken reminder process and multiple webpages of a website.

The at least one cellphone number and the at least one medicine taken reminding time are input by the user from operating the input device (27).

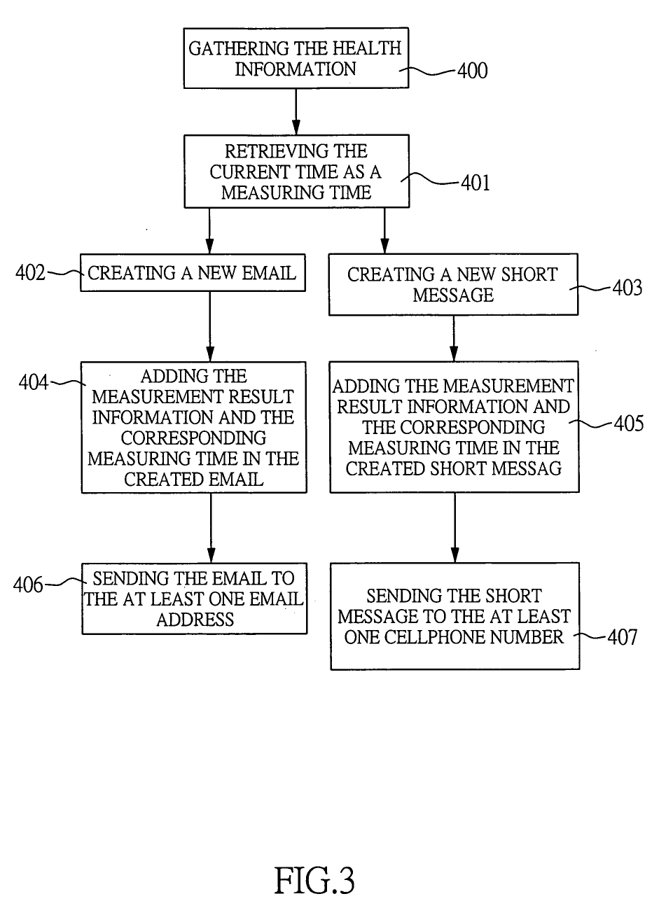

The measurement result information notification process is executed by the control unit (22) to send an email from the email program having the gathered measurement result information and the corresponding measuring time to the at least one email address via the network connecting unit (23). Furthermore, when the data gathering device (20) has the GPRS module (24), the control unit (22) will send a short message having the gathered measurement result information and the corresponding measuring time to the at least one cellphone number via the GPRS module (24). With further reference to FIG. 3, the measurement result information notification process may comprise steps of gathering the measurement result information (400), retrieving the current time as a measuring time (401), creating a new email (402) and a new short message (403) selectively or at the same time, adding the measurement result information and the corresponding measuring time in the created email after creating a new email (404), adding the measurement result information and the corresponding measuring time in the created short message after creating a new short message (405), sending the email to the at least one email address after adding the measurement result information and the corresponding measuring time in the created email (406) and sending the short message to the at least one cellphone number after adding the measurement result information and the corresponding measuring time in the created short message (407).

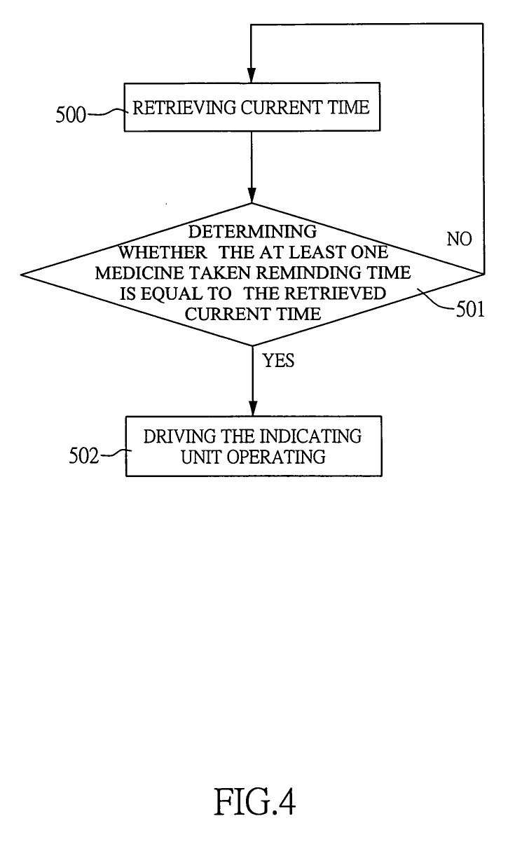

The medicine taken reminder process is executed by the control unit (22) to drive the indicating unit (28) operating, such as blinking the LEDs (281), outputting an alarm from the speaker (282) or the both, to remind the user that it is time to take medicine when the current time matches the at least one medicine taken reminding time. With further reference to FIG. 4, the medicine taken reminder process may comprise steps of retrieving current time (500), determining whether the at least one medicine taken reminding time is equal to the retrieved current time (501), driving the indicating unit operating when the at least one medicine taken reminding time is equal to the retrieved current time (502).

The webpages include the measurement result information and the corresponding measuring time stored in the memory (29) so the webpages show the measurement result information and the corresponding measuring time when the webpages are browsed.

The power module (202) is electrically connected and provides electric power to the control unit (22), the network connecting unit (23), the GPRS module (24), the thermometer (25), the hygrometer (26), the display (201), the indicating unit (28) and the memory (29). Furthermore, the power module (202) may be a battery mounted in the casing (200) or a power adapter having an electric plug for plugging in a power socket to obtain electric power.

The at least one electric appliance (30) is used to receive the email sent to the at least one email address and the short message sent to the at least one cellphone number stored in the memory (29) and connect to the website stored in the memory (29) to browse through the webpages of the website and shows the gathered measurement result information. Furthermore, the present invention may have multiple electric appliances (30), and each electric appliance (30) may be a desktop, a laptop, a cellphone or any other electric appliance (30) for receiving email or short message. In addition, the email and the short message sent by the control unit (22) when executing the measurement result information notification process may further have the Internet protocol (IP) address of the website stored in the memory (29) because the IP address of the website stored in the memory (29) is required for user to connect to the website with the at least one electric appliance (30).

The household health monitoring system as described does not require setting a server so anyone can easily construct the household health monitoring system by having at least one medical instrument (10) connected to the data gathering device (20) and having at least one electric appliance (30) used to receive the email or short message sent from the data gathering device (20) and connect to the website stored in the memory (29) to browse through the webpages of the website and show the gathered measurement result information. Therefore, household health monitoring system of the present invention is especially suitable for families having elder members. The families of the elder people only require to input email addresses and cellphone numbers to the data gathering device (20). After the medical instrument (10) completes measuring measurement result information of the elder people, the data gathering device (20) will send emails and short messages including the measurement result information to the email addresses and cellphone numbers stored in the data gathering device (20). Accordingly, the families of the elder people will easily know the measurement result information of the elder people at home and determine the health conditions of the elder people from the measurement result information. Furthermore, the data gathering device (20) provides a medicine taken reminder process to remind the user. That is, the control unit (22) determines whether the current time counted by the RTC (221) matches the at least one medicine taken reminding time according to the. If matched, the LEDs (281) is driven to glitter and the speaker (282) is driven to output alarm to remind the elder people. Therefore, the elder people do not forget to take medicine because of the reminder of the data gathering device (20) in the present invention.

Even though numerous characteristics and advantages of the present invention have been set forth in the foregoing description, together with details of the structure and function of the invention, the disclosure is illustrative only. Changes may be made in detail, especially in matters of arrangement of parts within the principles of the invention to the full extent indicated by the broad general meaning of the terms in which the appended claims are expressed.

What is claimed is:

1. A household health monitoring system comprising:
at least one medical instrument measuring a user's measurement result information;
a data gathering device storing at least one email address, electronically connected to the at least one medical instrument to gather the measurement result information and sending an email having the gathered measurement result information to the at least one email address; and
at least one electric appliance used to receive the email sent to the at least one email address and showing the gathered measurement result information.

2. The system as claimed in claim 1, wherein the data gathering device further comprises:
a casing;
at least one connecting interface mounted in and exposed through the casing to connect correspondingly to the at least one medical instrument;
a control unit mounted in the casing and electronically connected to the at least one medical instrument through the corresponding connecting interface to gather the measurement result information from the at least one medical instrument;
a network connecting unit mounted in the casing, electronically connected to the control unit and used to connect to the Internet;
an input device mounted on the casing and electronically connected to the control unit to allow the user to control and operate the data gathering device;
a display mounted on the casing and connected to the control unit to display the gathered measurement result information;
a memory mounted in the casing, electronically connected to the control unit to store the gathered measurement result information and storing
an electric mail (email) program;
at least one email address input by the user from operating the input device; and
a measurement result information notification process executed by the control unit to send an email having the gathered measurement result information from the email program to the at least one email address via the network connecting unit; and
a power module electrically connected and provides electric power to the control unit, the network connecting unit, the display and the memory.

3. The system as claimed in claim 2, wherein
the data gathering device further comprises a general packet radio service (GPRS) module mounted in the casing, electronically connected to the control unit and the power module and used to connect to a global system for mobile communications network;
the memory further stores at least one cellphone number input by the user from operating the input device;
the measurement result information notification process further send a short message having the gathered measurement result information to the at least one cellphone number via the GPRS module; and
the at least one electric appliance further used to receive short message sent to the at least one cellphone number.

4. The system as claimed in claim 3, wherein
the control unit further has a real-time clock (RTC) to count the current time;
the control unit further records a measuring time from the RTC when the control unit gathers the measurement result information from the at least one medical instrument, and the measuring time corresponds to the gathered measurement result information and is stored in the memory; and
the measurement result information notification process further adds the measuring time corresponding to the measurement result information in the email and the short message.

5. The system as claimed in claim 3, wherein
the memory further stores multiple webpages of a website, and the webpages have the measurement result information stored in the memory; and
the measurement result information notification process further adds the Internet protocol address of the website stored in the memory to the email and the short message.

6. The system as claimed in claim 4, wherein
the memory further stores multiple webpages of a website, and the webpages have the measurement result information and the corresponding measuring time stored in the memory; and
the measurement result information notification process further adds the Internet protocol address of the website stored in the memory to the email and the short message.

7. The system as claimed in claim 3, wherein
the data gathering device further comprises an indicating unit mounted on the casing, electronically connected to the control unit and the power module and controlled by the control unit;
the memory further stores
at least one medicine taken reminding time input by the user from operating the input device; and
a medicine taken reminder process driving the indicating unit operating to remind the user that it is time to take medicine when the current time matches the at least one medicine taken reminding time.

8. The system as claimed in claim 3, wherein
the data gathering device further comprises:
a thermometer mounted in the casing and electronically connected to the control unit and the power module to sense and send the indoor temperature in analog data form to the control unit; and
a hygrometer mounted in the casing and electronically connected to the control unit and the power module to sense and send the indoor humidity in analog data form to the control unit; and
the control unit further has analog to digital converting capability to convert analog data into digital data.

9. The system as claimed in claim 7, wherein the indicating unit further comprises multiple light emitting diodes electronically connected to and controlled by the control unit.

10. The system as claimed in claim 7, wherein the indicating unit further comprises a speaker electronically connected to and controlled by the control unit.

11. The system as claimed in claim 7, wherein the indicating unit further comprises multiple light emitting diodes and a speaker electronically both connected to and controlled by the control unit.

12. The system as claimed in claim 2, wherein the network connecting unit further comprises a physical wire connector exposing through the casing to connect to the Internet with a wire.

13. The system as claimed in claim 2, wherein the network connecting unit further comprises a wireless connector connecting to the Internet under wireless fidelity protocol.

14. The system as claimed in claim 2, wherein the network connecting unit further comprises:
a physical wire connector exposing through the casing to connect to the Internet with a wire; and
a wireless connector connecting to the Internet under wireless fidelity protocol.
